(19) United States
(12) Patent Application Publication
(10) Pub. No.: US 2025/0049357 A1
(43) Pub. Date: Feb. 13, 2025

(54) TISSUE PROFILE WELLNESS MONITOR

(71) Applicant: Masimo Corporation, Irvine, CA (US)

(72) Inventor: Marcelo M. Lamego, Cupertino, CA (US)

(21) Appl. No.: 18/929,360

(22) Filed: Oct. 28, 2024

Related U.S. Application Data
(63) Continuation of application No. 18/297,315, filed on Apr. 7, 2023, now Pat. No. 12,156,733.
Application No. 18/297,315 is a continuation of application No. 17/203,387, filed on Mar. 16, 2021, now Pat. No. 11,647,923.
Application No. 17/203,387 is a continuation of application No. 16/286,393, filed on Feb. 26, 2019, now Pat. No. 10,980,457.
Application No. 16/286,393 is a continuation of application No. 15/814,229, filed on Nov. 15, 2017, now Pat. No. 10,251,586.
Application No. 15/814,229 is a continuation of application No. 14/599,342, filed on Jan. 16, 2015, now Pat. No. 9,848,807.
Application No. 14/599,342 is a continuation of application No. 13/764,007, filed on Feb. 11, 2013, now Pat. No. 8,965,471.
Application No. 13/764,007 is a continuation of application No. 12/106,969, filed on Apr. 21, 2008, now Pat. No. 8,374,665.
(60) Provisional application No. 60/925,811, filed on Apr. 21, 2007.

(51) Int. Cl. A61B 5/1455; A61B 5/00; A61B 5/024; A61B 5/0295

(52) U.S. Cl. CPC A61B 5/14551; A61B 5/02416; A61B 5/0295; A61B 5/72; A61B 5/7275; A61B 5/7405; A61B 5/742; A61B 5/746; A61B 5/7475

(57) ABSTRACT

A tissue profile wellness monitor measures a physiological parameter, generates a tissue profile, defines limits and indicates when the tissue profile exceeds the defined limits. The physiological parameter is responsive to multiple wavelengths of optical radiation after attenuation by constituents of pulsatile blood flowing within a tissue site. The tissue profile is responsive to the physiological parameter. The limits are defined for at least a portion of the tissue profile.

### CROSS-REFERENCE TO RELATED APPLICATIONS

The present application is a continuation of U.S. patent application Ser. No. 18/297,315, filed on Apr. 7, 2023, entitled “TISSUE PROFILE WELLNESS MONITOR,” which is a continuation of U.S. patent application Ser. No. 17/203,387, filed on Mar. 16, 2021, entitled “TISSUE PROFILE WELLNESS MONITOR,” which is a continuation of U.S. patent application Ser. No. 16/286,393, filed Feb. 26, 2019, entitled “TISSUE PROFILE WELLNESS MONITOR,” which is a continuation of U.S. patent application Ser. No. 15/814,229, filed Nov. 15, 2017, entitled “TISSUE PROFILE WELLNESS MONITOR,” which is a continuation of U.S. patent application Ser. No. 14/599,342, filed Jan. 16, 2015, entitled “TISSUE PROFILE WELLNESS MONITOR,” which is a continuation of U.S. patent application Ser. No. 13/764,007, filed Feb. 11, 2013, entitled “TISSUE PROFILE WELLNESS MONITOR,” which is a continuation of U.S. patent application Ser. No. 12/106,969, filed Apr. 21, 2008, entitled “TISSUE PROFILE WELLNESS MONITOR,” which claims priority benefit under 35 U.S.C. § 119 (e) to U.S. Provisional Application Ser. No. 60/925,811, filed Apr. 21, 2007, entitled “TISSUE PROFILE WELLNESS MONITOR.” All of the above referenced applications are hereby incorporated by reference in their entirety herein.

### BACKGROUND OF THE INVENTION

Spectroscopy is a common technique for measuring the concentration of organic and some inorganic constituents of a solution. The theoretical basis of this technique is the Beer-Lambert law, which states that the concentration cᵢ of an absorbent in solution can be determined by the intensity of light transmitted through the solution, knowing the path length d_{λ}, the intensity of the incident light I_{0,λ}, and the extinction coefficient ϵ_{i,λ} at a particular wavelength λ. In generalized form, the Beer-Lambert law is expressed as:

$${I}_{λ} = {I}_{0 , λ} {e}^{- {d}_{λ} \cdot {μ}_{a , λ}} \qquad ( 1 )$$

$${μ}_{a , λ} = {\sum }_{i = 1}^{n} {ε}_{i , λ} \cdot {c}_{i} \qquad ( 2 )$$

Where μ_{a,80} is the bulk absorption coefficient and represents the probability of absorption per unit length. The minimum number of discrete wavelengths that are required to solve EQS. 1-2 are the number of significant absorbers that are present in the solution.

A practical application of this technique is pulse oximetry, which utilizes a noninvasive sensor to measure oxygen saturation (Sp0₂) and pulse rate. The sensor has light emitting diodes (LEDs) that transmit optical radiation of red and infrared wavelengths into a tissue site and a detector that responds to the intensity of the optical radiation after attenuation by pulsatile arterial blood flowing within the tissue site. Based on this response, a processor determines measurements for Sp0₂ and pulse rate, and outputs representative plethysmographic waveforms. Thus, “pulse oximetry” as used herein encompasses its broad ordinary meaning known to one of skill in the art, which includes at least those noninvasive procedures for measuring parameters of circulating blood through spectroscopy. Moreover, “plethysmograph” as used herein encompasses its broad ordinary meaning known to one of skill in the art, which includes at least data representative of a change in the absorption of particular wavelengths of light as a function of the changes in body tissue resulting from pulsing blood.

Pulse oximeters capable of reading through motion induced noise are available from Masimo Corporation (“Masimo”) of Irvine, California. Moreover, portable and other oximeters capable of reading through motion induced noise are disclosed in at least U.S. Pat. Nos. 6,770,028, 6,658,276, 6,584,336, 6,263,222, 6,157,850, 5,769,785, and 5,632,272, which are owned by Masimo, and are incorporated by reference herein. Such reading through motion oximeters have gained rapid acceptance in a wide variety of medical applications, including surgical wards, intensive care and neonatal units, general wards, home care, physical training, and virtually all type of monitoring scenarios.

FIG. 1 illustrates an absorption graph 100 having a dimensionless vertical axis 101 of relative light absorption and a horizontal axis 102 of transmitted wavelength in nm. Shown is a plot of Hb0₂ absorption 110 and Hb absorption 120 versus wavelength, both normalized to the absorption at 800 nm. At red and near IR wavelengths below 970 nm, where water has a significant peak, Hb and Hb0₂ are the only significant absorbers normally present in the blood. Thus, typically only two wavelengths are needed to resolve the concentrations of Hb and Hb0₂, e.g. a red (RD) wavelength at 660 nm and an infrared (IR) wavelength at 940 nm. In particular, Sp0₂ is computed based upon a red ratio Red_{AC}/Red_{DC} and an IR ratio IR_{AC}/IR_{DC}, which are the AC detector response magnitude at a particular wavelength normalized by the DC detector response at that wavelength. The normalization by the DC detector response reduces measurement sensitivity to variations in tissue thickness, emitter intensity and detector sensitivity, for example. The AC detector response is a plethysmograph, as described above. Thus, the red and IR ratios can be denoted as NP_{RD} and NP_{IR} respectively, where NP stands for “normalized plethysmograph.” In pulse oximetry, oxygen saturation is calculated from the ratio NP_{RD}/NP_{IR}.

### SUMMARY OF THE INVENTION

Oxygen saturation is a very useful physiological parameter for indicating the cardiovascular status of a patient, but allows healthcare providers only a few minutes warning that a patient is potentially having a medical crisis. A wellness indicator advantageously monitors changes in a patient's “tissue profile” so as to provide an advance warning of a deteriorating medical condition. This tissue profile is provided by a multiple wavelength sensor and a noninvasive multi-parameter patient monitor, which make blood absorption measurements at more than a red wavelength and an IR wavelength of conventional pulse oximetry. In one embodiment, described below, blood absorption measurements are made at eight wavelengths. Advantageously, this rich wavelength data characterizes a tissue site over a wavelength spectrum.

FIG. 2 illustrates an example of a tissue profile. In this example, the sensor emits eight wavelengths (610, 620, 630, 655, 700, 720, 800 and 905 nm). A tissue profile graph 200 has a NP ratio axis 201 and a wavelength axis 202, where the NP ratios are of the form NP_{λ1}/NP_{λ2}. This is a generalization to multiple wavelengths of the ratio NP_{RD}/NP_{IR} described above for two (red and IR) wavelengths. In order to provide a common scale for these NP ratios, the ratios are calculated with respect to a reference wavelength, λr, which may be any of the available wavelengths. Thus, the plotted NP ratios 210 are denoted NP_{λn}/NP_{λr}. Note that the NP ratio at the reference wavelength is NP_{λr}/NP_{λr}=1, which is 700 nm in this example. In this example, a tissue profile 210 is plotted for Sp02=97%.

Not surprisingly, the tissue profile 210 has the same general shape as the absorption curves 110, 120 of FIG. 1. In particular, the AC component of the detector signal relative to the DC component (NP) for a specific wavelength is proportional to the light absorption at that wavelength. Thus, the NP ratio magnitudes and hence the points along a tissue profile curve are proportional to absorption. Assuming negligible abnormal Hb species, if SpO₂ is close to 100%, most of the absorption is due to HbO₂ and, accordingly, the tissue profile is shaped closely to the HbO₂ absorption curve. As SpO₂ decreases from 100%, the tissue profile shape is increasing influenced by the shape of the Hb absorption curve.

In one embodiment, the tissue profile 210 consists solely of the measured NP ratios at the sensor wavelengths, i.e. a finite set of discrete values. In another embodiment, the tissue profile 210 consists of the measured NP ratios and defined NP ratio values between the sensor wavelengths, which are based upon tissue absorption characteristics. That is, the tissue profile 210 is a curve defined over a continuous range of wavelengths, including the sensor wavelengths. Although described above with respect to NP ratios derived from the AC component of the detector signal, a DC tissue profile may also be defined and applied to patient monitoring, as described below.

A tissue profile or tissue characterization is described in U.S. patent application Ser. No. 11/367,034, filed Mar. 1, 2006 entitled Physiological Parameter Confidence Measure; a multiple wavelength sensor is disclosed in U.S. patent application Ser. No. 11/367,013, filed Mar. 1, 2006 entitled Multiple Wavelength Sensor Emitters; and a multi-parameter patient monitor is disclosed in U.S. patent application Ser. No. 11/367,033, filed Mar. 1, 2006 entitled Noninvasive Multi-Parameter Patient Monitor, all of the aforementioned applications are assigned to Masimo Laboratories, Inc., Irvine, CA and all are incorporated by reference herein.

One aspect of a tissue profile wellness monitor comprises generating a tissue profile, predetermining rules and applying the rules to the tissue profile. The tissue profile is responsive to absorption of emitted wavelengths of optical radiation by pulsatile blood flowing within a tissue site. The rules are used to evaluate at least a portion of the tissue profile. A patient condition is indicated according to the applied rules.

Another aspect of a tissue profile wellness monitor comprises measuring a normalized plethysmograph (NP) to generate a tissue profile, testing the tissue profile and outputting the test results. The NP is measured at each of multiple wavelengths of optical radiation, and the NP is responsive to attenuation of the optical radiation by constituents of pulsatile blood flowing within a tissue site illuminated by the optical radiation. The tissue profile is tested against predetermined rules. The test results are output as at least one of a display, alarm, diagnostic and control.

A further aspect of a tissue profile wellness monitor comprises measuring a physiological parameter, generating a tissue profile, defining limits and indicating when the tissue profile exceeds the defined limits. The physiological parameter is responsive to multiple wavelengths of optical radiation after attenuation by constituents of pulsatile blood flowing within a tissue site. The tissue profile is responsive to the physiological parameter. The limits are defined for at least a portion of the tissue profile.

### BRIEF DESCRIPTION OF THE DRAWINGS

FIG. 1 is a graph of oxyhemoglobin and reduced hemoglobin light absorption versus wavelength across portions of the red and IR spectrum;

FIG. 2 is a graph of normalized plethysmograph (NP) ratios versus wavelength illustrating a tissue profile for 97% oxygen saturation;

FIG. 3 is a general block diagram of a patient monitoring system embodiment that implements a tissue profile wellness monitor;

FIG. 4 is a graph of tissue profiles for high saturation, low saturation, high carboxyhemoglobin (HbCO) and high methemoglobin (MetHb);

FIG. 5 is a graph illustrating tissue profile changes indicative of patient wellness; and

FIG. 6 is a block diagram of a tissue profile wellness monitor embodiment.

### DETAILED DESCRIPTION OF THE PREFERRED EMBODIMENT

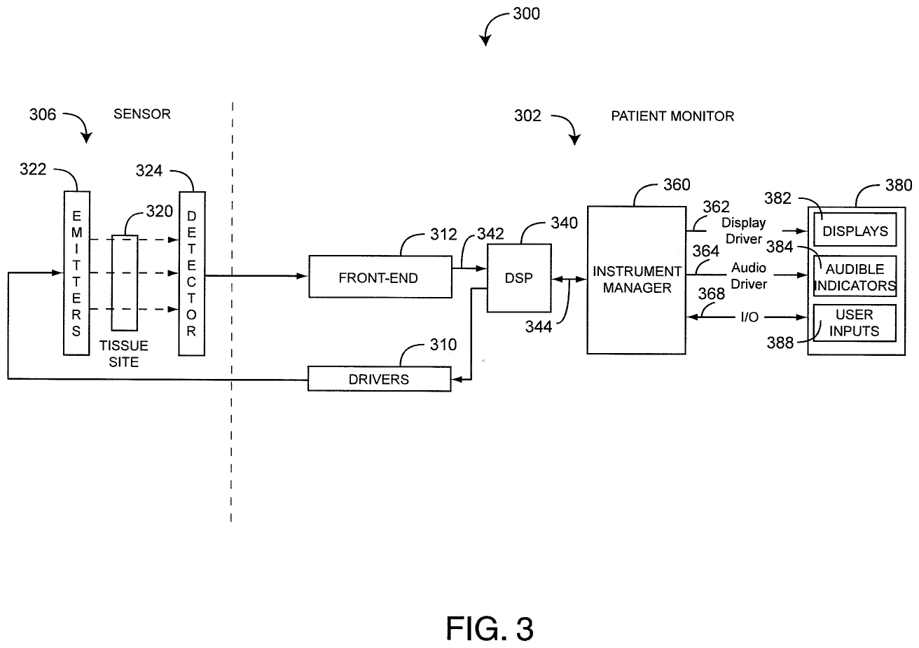

FIG. 3 illustrates a patient monitoring system 300, which generates NP ratios and blood parameter measurements, such SpO₂, HbCO and HbMet, accordingly. The patient monitoring system is advantageously adapted as a tissue profile wellness monitor, as described below. The patient monitoring system 300 has a patient monitor 302 and a sensor 306. The sensor 306 attaches to a tissue site 320 and includes a plurality of emitters 322 capable of irradiating the tissue site 320 with differing wavelengths of light, perhaps including the red and infrared wavelengths utilized in pulse oximeters. The sensor 306 also includes one or more detectors 324 capable of detecting the light after attenuation by the tissue site 320. A multiple wavelength sensor is disclosed in U.S. application Ser. No. 11,367,013, filed on Mar. 1, 2006, titled Multiple Wavelength Sensor Emitters, cited above. Multiple wavelength sensors, such as Rainbow™-brand adhesive and reusable sensors are available from Masimo Corporation, Irvine, California.

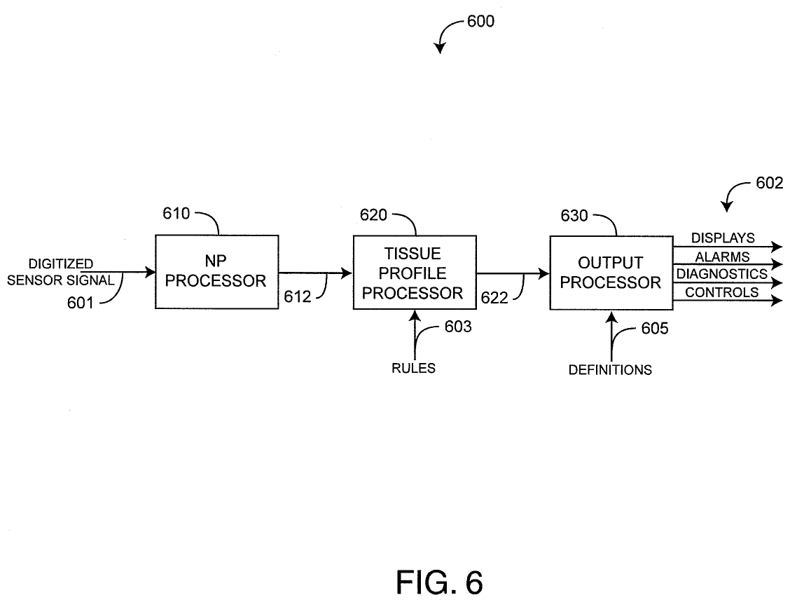

As shown in FIG. 3, the patient monitor 302 communicates with the sensor 306 to receive one or more intensity signals indicative of one or more physiological parameters. Drivers 310 convert digital control signals into analog drive signals capable of driving the sensor emitters 322. A front-end 312 converts composite analog intensity signal(s) from light sensitive detector(s) 324 into digital data 342 input to the DSP 340. The DSP 340 may comprise a wide variety of data and/or signal processors capable of executing programs for determining physiological parameters from input data. In an embodiment, the DSP 340 executes the processors 610, 620, 630 (FIG. 6), described below.

The instrument manager 360 may comprise one or more microcontrollers providing system management, such as monitoring the activity of the DSP 340. The instrument manager 360 also has an input/output (I/O) port 368 that provides a user and/or device interface for communicating with the monitor 302. In an embodiment, the I/O port 368 provides threshold settings via a user keypad, network, computer or similar device, as described below.

Also shown in FIG. 3 are one or more user I/O devices 380 including displays 382, audible indicators 384 and user inputs 388. The displays 382 are capable of displaying indicia representative of calculated physiological parameters such as one or more of a pulse rate (PR), plethysmograph (pleth), perfusion index (PI), signal quality and values of blood constituents in body tissue, including for example, oxygen saturation (SpO₂), carboxyhemoglobin (HbCO) and methemoglobin (HbMet). The monitor 302 may also be capable of storing or displaying historical or trending data related to one or more of the measured parameters or combinations of the measured parameters. The monitor 302 may also provide a trigger for the audible indictors 384, which operate beeps, tones and alarms, for example. Displays 382 include for example readouts, colored lights or graphics generated by LEDs, LCDs or CRTs to name a few. Audible indicators 384 include speakers or other audio transducers. User input devices 388 may include, for example, keypads, touch screens, pointing devices, voice recognition devices, or the like.

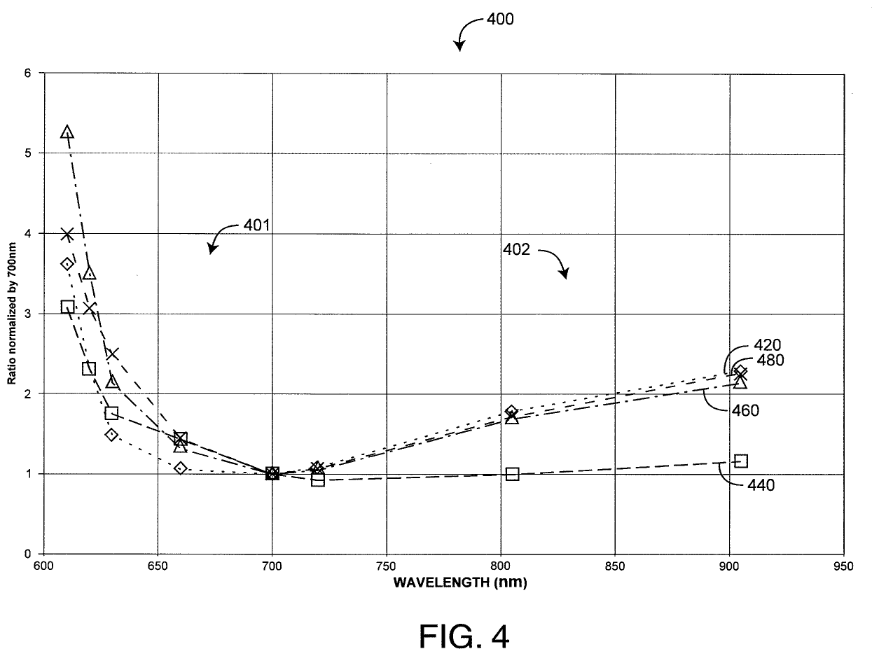

FIG. 4 illustrates tissue profile curves 400, which are responsive to Hb constituents. In this example, the sensor emits eight wavelengths (610, 620, 630, 660, 700,720, 805, 905 nm), which are normalized at 700 nm. Shown is a high saturation profile curve 420, e.g. Sp02≈100% (⋄); a low saturation profile curve 440, e.g. Sp02=70% (□); a high HbCO profile curve 460, e.g. HbCO=30% (Δ); and a high HbMet profile curve 480, e.g. HbMet=6% (×). The profile curves 420-480 each has a head portion 401 at wavelengths less than 700 nm and a corresponding tail portion 402 at wavelengths of greater than 700 nm. As shown in FIG. 4, a tissue profile head portion 401 has higher values when HbCO (Δ) or HbMet (×) has a higher percentage value. The head portion 401 has lower values when HbCO or HbMet has a lower percentage value. Also, both the head portion 401 and the tail portion 402 have higher values when Sp02 is a high percentage (⋄) and lower values when Sp02 is a low percentage (□).

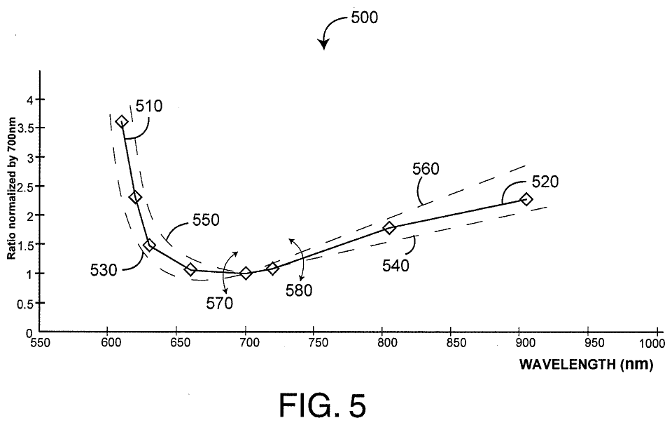

FIG. 5 illustrates an example tissue profile 500 utilized as a wellness indicator. As described with respect to FIG. 4 above, the position or shape of the tissue profile or changes in the position or shape of the tissue profile provide an indication of patient wellness. In particular, position, shape or relative movements of the curve “head” 510 or the curve “tail” 520 or both indicate potentially detrimental values or changes in values of hemoglobin constituents. For example, a drop in the tissue profile head 510 or tail 520 below a predefined boundary 530, 540 may indicate reduced oxygen saturation. As another example, a rise in the tissue profile head 510 above a predefined boundary 550 may indicate increased concentrations of abnormal hemoglobin species, such as carboxyhemoglobin (HbCO) and methemoglobin (HbMet). Further, relative movements 570, 580 of the tissue profile 500 faster than a predefined rate may indicate potentially serious trends in the concentrations of normal or abnormal hemoglobin species.

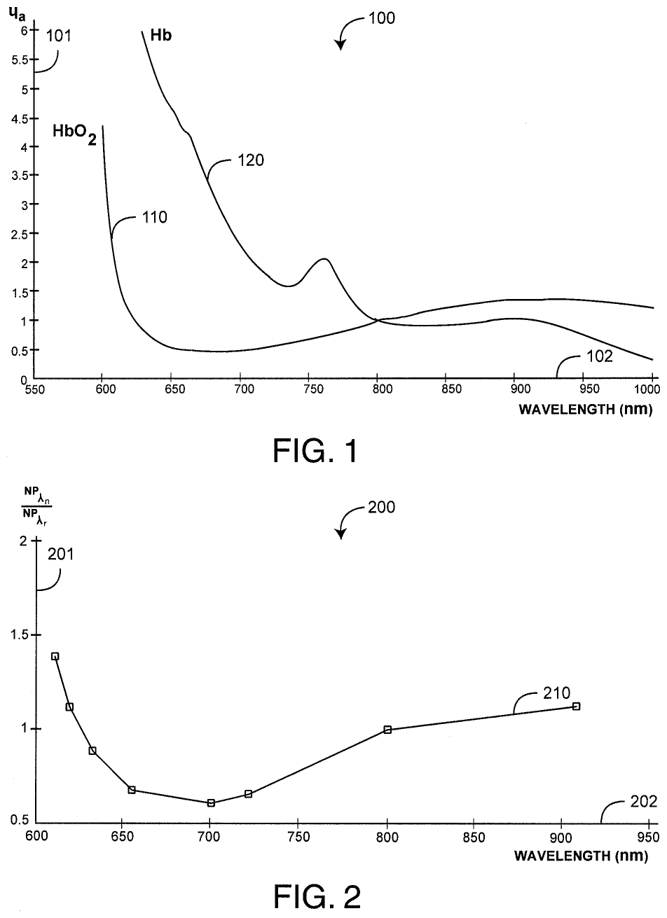

FIG. 6 illustrates a tissue profile wellness monitor 600 having a NP processor 610, a tissue profile processor 620 and an output processor 630. In an embodiment, these processors 610-630 execute in the DSP 340 (FIG. 3) to monitor tissue profile changes. The NP processor 610 has digitized sensor signal input 601 from one or more sensor channels, such as described with respect to FIG. 3, above, and generates normalized plethysmograph (NP) calculations 612 as described with respect to FIG. 1, above.

As shown in FIG. 6, the tissue profile processor 620 is configured to compare tissue profile changes 612 with respect to predetermined rules 603 and communicate the test results 622 to the output processor 630. As an example, the tissue profile processor 620 may communicate to the output processor 630 when a tissue profile portion changes faster than a predetermined rate.

Also shown in FIG. 6, the output processor 630 inputs the tissue profile processor results 622 and generates outputs 602 based upon predetermined output definitions 605. For example, if a test profile result is “true”, it might trigger an audible alarm. Rules and corresponding outputs are described in further detail with respect to TABLE 1, below.

In an embodiment, the tissue profile wellness monitor 600 provides outputs 602 according to TABLE 1, below. The terms listed in TABLE 1 are described with respect to FIG. 6, above. Various other indicators, alarms, controls and diagnostics in response to various combinations of rules and output definitions can be substituted for, or added to, the rule-based outputs illustrated in TABLE 1.

In an embodiment, the tissue profile wellness monitor 600 grades a patient with respect to wellness utilizing green, yellow and red indicators. For example, a green panel light signals that the tissue profile is indicative of normal blood hemoglobin. A yellow panel light signals that changes in the tissue profile shape or position are indicative of potentially problematic changes in blood hemoglobin. A red panel light signals that the tissue profile is indicative of blood hemoglobin outside of normal ranges.

TABLE 1
Tissue Profile Rules and Outputs

| TISSUE PROFILE RULES | OUTPUTS |
|---|---|
| If all portions of tissue profile are | Then illuminate green indicator. |
| within boundaries and relatively | |
| unchanging over time | |
| If tail drops faster than tail trend | Then illuminate yellow indicator |
| limit; or head rises faster than head | |
| trend limit | |
| If tail or head are outside of boundaries | Then illuminate red indicator |

A tissue profile wellness monitor has been disclosed in detail in connection with various embodiments. These embodiments are disclosed by way of examples only and are not to limit the scope of the claims that follow. One of ordinary skill in art will appreciate many variations and modifications.

1. (canceled)

2. A physiological monitoring method comprising:
receiving, from a sensor comprising a light source that emits light into a tissue site of a patient and a detector that detects a plurality of wavelengths of the emitted light after attenuation by the tissue site, a signal indicative of the detected light at each of the plurality of wavelengths;
determining a tissue profile curve across each of the plurality of wavelengths based at least in part on the signal indicative of the detected light at each of the plurality of wavelengths;
comparing the tissue profile curve to a predefined boundary curve to determine patient wellness, wherein the predefined boundary curve is defined across each of the plurality of wavelengths; and
outputting an indication in response to determining the tissue profile curve exceeds the predefined boundary curve.

3. The method of claim 2, wherein the indication comprises at least one of a display, an alarm, a diagnostic, or a control.

4. The method of claim 2, wherein determining the tissue profile curve comprises, at least in part:
measuring, from the signal, values indicative of an amount of light attenuation for at least some of the plurality wavelengths;
defining, based on characteristics of the tissue site, additional values corresponding to at least a portion of a wavelength spectrum between the respective wavelengths associated with the at least some of the plurality of wavelengths; and
combining the measured values at each of the at least some of the plurality wavelengths with the additional values.

5. The method of claim 2 further comprising:
outputting a first signal indicative of patient normalcy when the tissue profile curve is within the predefined boundary curve and a rate of change of the tissue profile curve is less than a maximum rate of change; and
outputting a second signal indicative of alert when at least a portion of the tissue profile curve is outside of the predefined boundary curve and the rate of change of the tissue profile curve is greater than the maximum rate of change.

6. The method of claim 2, wherein the tissue profile curve is indicative of oxygen saturation of the patient.

7. The method of claim 2, wherein the tissue profile curve is indicative of at least one of; carboxyhemoglobin of the patient, or methemoglobin of the patient.

8. The method of claim 2, wherein the tissue profile curve comprises a first portion and a second portion, and wherein the first portion is analyzed in comparison to a first portion predefined boundary curve, and the second portion is analyzed in comparison to a second portion predefined boundary curve.

9. The method of claim 8 further comprising:
providing a first indication in response to the first and second portions not exceeding the respective first and second portion predefined boundary curves;
providing a second indication in response to one of the first or second portions exceeding the respective first or second portion predefined boundary curves; and
providing a third indication in response to both of the first and second portions exceeding the respective first and second portion predefined boundary curves.

10. The method of claim 2, wherein determining the tissue profile curve comprises, at least in part:
determining, at each of the plurality of wavelengths and based on the signal, normalized plethysmographs (NPs); and
normalize each of the determined NPs based on a reference wavelength.

11. The method of claim 10, wherein the reference wavelength is one of the plurality of wavelengths.

12. A physiological monitoring method comprising:
receiving, from a sensor comprising a light source that emits light into a tissue site of a patient and a detector that detects a plurality of wavelengths of the emitted light after attenuation by the tissue site, a signal indicative of the detected light at each of the plurality of wavelengths;
determining a plurality of tissue profile curves based at least in part on signals received from the sensor indicative of detected light at each of a plurality of wavelengths, wherein each of the tissue profile curves is across each of the plurality of wavelengths;
evaluating a trend in movement of the tissue profile curves over time to determine patient wellness; and
outputting an indication of the determined patient wellness.

13. The method of claim 12, wherein the trend in movement of the tissue profile curve exceeding a predetermined trend limit indicates advance warning of deteriorating patient wellness.

14. The method of claim 12, wherein determining the tissue profile curve comprises, at least in part:
determining, at each of the plurality of wavelengths and based on the signal, normalized plethysmographs (NPs); and
normalizing each of the determined NPs based on a reference wavelength.

15. The method of claim 14, wherein the reference wavelength is one of the plurality of wavelengths.

16. The method of claim 14, wherein determining the tissue profile curve further comprises, at least in part:
defining continuous normalized NP values corresponding to at least a portion of a wavelength spectrum between the two or more of the plurality of wavelengths based at least in part on characteristics of the tissue site; and
combining the normalized NP values at each of the plurality of wavelengths with the defined continuous normalized NP values.

17. The method of claim 14, wherein the reference wavelength divides the tissue profile curve into a first portion and a second portion, and wherein the first portion is evaluated in relation to a first portion predetermined trend limit, and the second portion is evaluated in relation to a second portion predetermined trend limit.

18. The method of claim 17 further comprising:
providing a first indication in response to the first and second portions not exceeding the respective first and second portion predetermined trend limits;
providing a second indication in response to one of the first or second portions exceeding the respective first or second portion predetermined trend limits; and
providing a third indication in response to both of the first and second portions exceeding the respective first and second portion predetermined trend limits.

19. The method of claim 12, wherein the tissue profile curve is indicative of oxygen saturation of the patient.

20. The method of claim 12, wherein the tissue profile curve is indicative of at least one of: carboxyhemoglobin of the patient, or methemoglobin of the patient.
